(19) United States
(12) Patent Application Publication
(10) Pub. No.: US 2026/0096753 A1
(43) Pub. Date: Apr. 9, 2026

(54) Medical Extremity Warming System

(71) Applicants: Michele Duval, Prosperity, SC (US); Jeremy Duval, Prosperity, SC (US)

(72) Inventors: Michele Duval, Prosperity, SC (US); Jeremy Duval, Prosperity, SC (US)

(21) Appl. No.: 18/909,447

(22) Filed: Oct. 8, 2024

(51) Int. Cl. A61B 5/15

(52) U.S. Cl. CPC A61B 5/150076

(57) ABSTRACT

A medical extremity warming system includes a sleeve, a releasable securing assembly, and a heating assembly. The sleeve is wrappable around an extremity of a patient and configured to retain heat therein. The releasable securing assembly includes a first mating member at one side of the sleeve and a second mating member at a second side of the sleeve. The heating assembly is designed to supply heat and warm an extremity of a patient wrapped in the sleeve to a temperature sufficient to render veins more prominent and visible to facilitate insertion of an intravenous device. The heating assembly includes an air channel, a heating unit operatively connected to the air channel, and an exit port disposed on an interior surface of the sleeve and connected to the air channel. Alternatively, the heating assembly includes a plurality of oxygen-activated heating elements removably or permanently mounted on or in the sleeve.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to medical warming devices and more particularly pertains to a new medical extremity warming system for warming the extremity of a patient, such as an arm or leg, to a temperature sufficient to render veins more prominent and visible to facilitate insertion of a needle of an intravenous (IV) device, such as a catheter, IV fluid drip, or syringe for blood draws or injections.

### (2) Description of Related Art Including Information Disclosed Under 37 CFR 1.97 and 1.98

The prior art relates to medical warming devices. The prior art, as best understood, does not disclose an arm or leg heating system for intravenous therapy that is comprised of a heating unit and a disposable arm encircling sleeve which is heated by the heating unit and used to raise the temperature of a patient's arm to facilitate the insertion of a needle of an intravenous (IV) device.

### BRIEF SUMMARY OF THE INVENTION

An embodiment of the disclosure meets the needs presented above in a medical extremity warming system generally comprising a sleeve, a releasable securing assembly, and a heating assembly. The sleeve is wrappable around an extremity of a patient and configured to retain heat therein. The releasable securing assembly includes a first mating member at one side of the sleeve and a second mating member at a second side of the sleeve. The heating assembly is designed to supply heat and warm an extremity of a patient wrapped in the sleeve to a temperature sufficient to render veins more prominent and visible to facilitate insertion of an intravenous device.

In one possible embodiment, the heating assembly includes an air channel, a heating unit operatively connected to the air channel to conduct heated air into the air channel, and an exit port disposed on an interior surface of the sleeve and connected to the air channel to permit flow of heated air out of the exit port and about an extremity of a patient wrapped in the sleeve. In an alternative embodiment, the heating assembly includes a plurality of oxygen-activated heating elements removably or permanently mounted on or in the sleeve.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a medical extremity warming system according to an embodiment of the disclosure.

FIG. 2 is a bottom view of an embodiment of the disclosure.

FIG. 3 is a top view of an embodiment of the disclosure.

FIG. 4 is a top view of an embodiment of the disclosure in use.

FIG. 5 is a perspective view of an alternative embodiment of the disclosure.

FIG. 6 is a perspective view of an alternative embodiment of the disclosure.

### DETAILED DESCRIPTION OF THE INVENTION

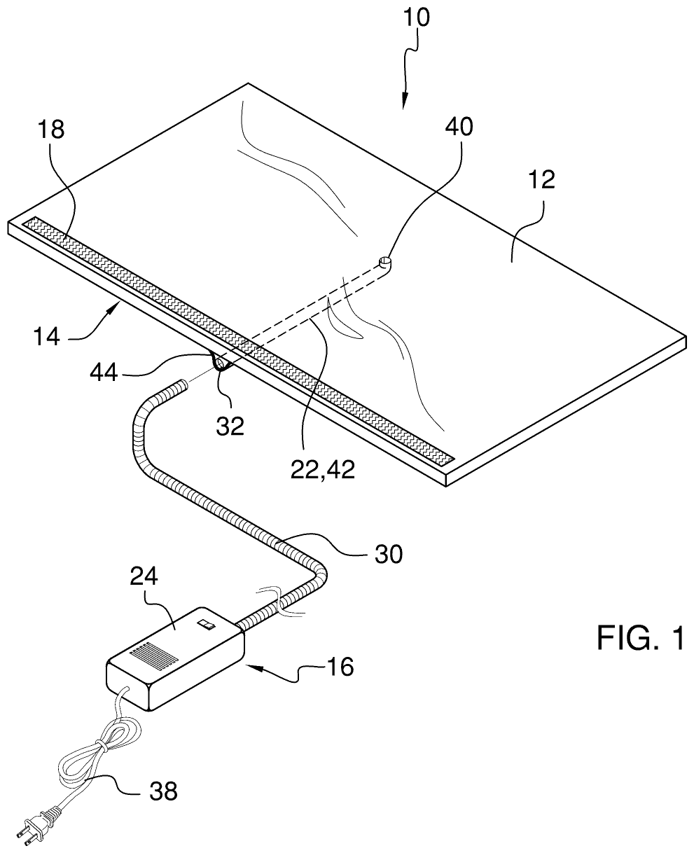

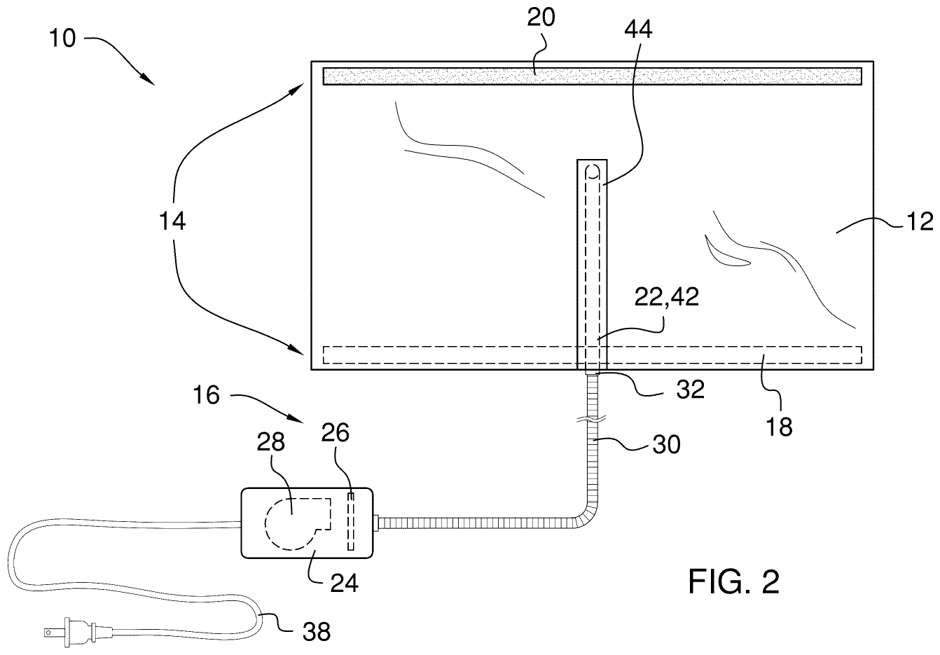

With reference now to the drawings, and in particular to FIGS. 1 through 6 thereof, a new medical extremity warming system embodying the principles and concepts of an embodiment of the disclosure and generally designated by the reference numeral 10 will be described.

As best illustrated in FIGS. 1 through 6, the medical extremity warming system 10 generally comprises a sleeve 12, a releasable securing assembly 14, and a heating assembly 16. The sleeve 12 is wrappable around an extremity of a patient. The releasable securing assembly 14 includes a first mating member 18 at one side of the sleeve 12 and a second mating member 20 at a second side of the sleeve 12. The heating assembly 16 is designed to heat the sleeve 12 to a temperature sufficient to warm an extremity of a patient to thereby render veins more prominent and visible to facilitate insertion of a needle of an intravenous device.

FIGS. 1 through 4 show one possible embodiment of the medical extremity warming system 10, wherein the heating assembly 16 includes an air channel 22 and a heating unit 24 operatively connected to the air channel 22 to conduct heated air into the air channel 22. An exit port 40 is disposed on an interior surface of the sleeve 12 and is connected to the air channel 22 to permit flow of heated air out of the exit port 40 and about an extremity of a patient wrapped in the sleeve 12. The heating unit 24 includes a heating element 26, a blower unit 28, and a hose 30 connecting the powered heating unit 24 to the air channel 22. The air channel 22 is in the form of a tube 42 that is located at an outer surface of the sleeve 12. The tube 42 has a connecting port 32 to detachably connect to the hose 30. In the exemplary embodiment shown, the exit port 40 is located near a central or middle portion of the sleeve 12 to promote better distribution of air throughout the sleeve 12. In the exemplary embodiment shown, a tube cover 44 holds the tube 42 in place on the sleeve 12, which tube cover 44 could be a natural or artificial fabric stitched or adhered to the sleeve 12, or possibly a resilient elastic material. In one possible embodiment, the tube 42 could be a plurality of tubes 42 in the form of a network of branches connected to a main line and a plurality of exit ports 40 spaced at intervals on the sleeve 12 to diffuse air throughout the sleeve 12. The heating unit 24 can either be a portable or handheld unit as shown in the exemplary embodiment or could be a wall-mounted unit or supported on a cart or stand. The heating unit 24 can be of various sizes and produce varying temperatures of heated air. The hose 30 can be of varying diameters to provide increased or decreased flow cross-section.

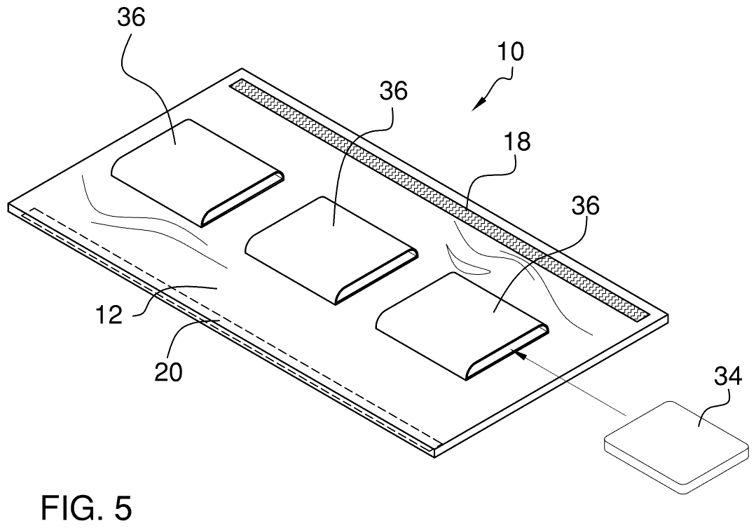

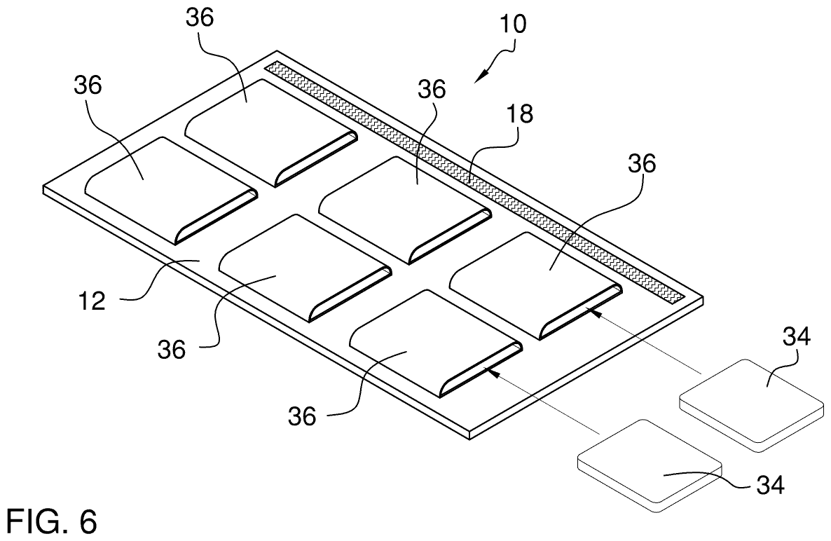

FIGS. 5 and 6 show an alternative possible embodiment of the medical extremity warming system 10, wherein the heating assembly 16 includes a plurality of oxygen-activated heating elements 34 removably or permanently mounted on or in the sleeve 12. In the embodiment shown in FIG. 5, the sleeve 12 includes three pockets 36 designed to each hold one of the heating elements 34 therein. In FIG. 6, the sleeve 12 includes six of the pockets 36. It should be understood that FIGS. 5 and 6 are exemplary embodiments, and the sleeve 12 could include any number of pockets 36 of any dimensions and in any configuration or placement as desired to produce a desired heating effect.

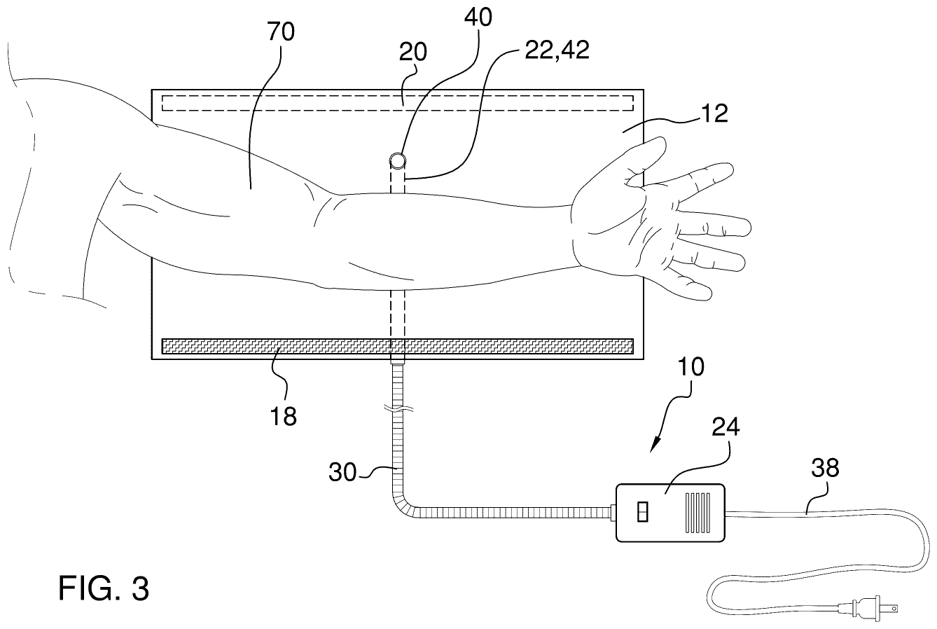

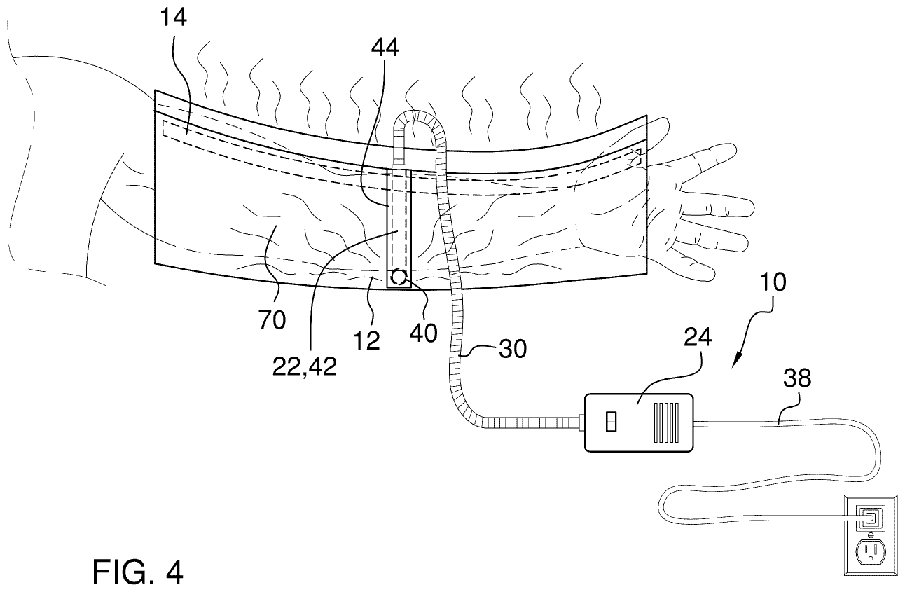

In either embodiment, the sleeve 12 can be made of a disposable material, such that the sleeve 12 is discarded after use. In the exemplary embodiment shown in FIGS. 3 and 4, the sleeve 12 has a width sufficient to wrap around an arm of a patient and a length sufficient to extend from a shoulder region to a hand region. However, the sleeve 12 could be sized as desired at manufacture to fit an entire arm or portions thereof, or an entire leg or portions thereof. For the securing assembly 14, the first mating member 18 includes a strip of hook material positioned along an edge of the sleeve 12 and the second mating member 20 includes a strip of loop material positioned along an opposite edge of the sleeve 12. However, straps, clips, buttons, zippers, or essentially any other mating or connecting structures could be utilized.

The medical extremity warming system 10 is used to prepare an extremity of a patient for insertion of an intravenous device. Heating an extremity can cause veins to become more prominent and visible, thereby making it much easier for medical personnel to find a suitable vein and insert a needle of an IV device, such as a catheter, IV fluid drip, or syringe for blood draws or injections. With reference to FIGS. 3 and 4, the medical personnel first wraps the sleeve 12 relatively tightly around an extremity of a patient, in this case an arm 70, and then releasably connects the first mating member 18 and the second mating member 20 to secure the sleeve 12 in place. The heating assembly 16 is activated to heat the sleeve 12 to a temperature sufficient to warm the arm 70, in this case by blowing heated air 46 out of the exit port 40 and throughout the interior of the sleeve 12 and around the arm 70. The medical personnel then waits a selected amount of time before releasing and unwrapping the sleeve 12. The medical personnel now has quite visible veins into which a needle of an intravenous device can be inserted.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A medical extremity warming system comprising:
a sleeve being wrappable around an extremity of a patient and configured to retain heat therein;
a releasable securing assembly comprising a first mating member at one side of said sleeve and a second mating member at a second side of said sleeve; and
a heating assembly being configured to supply heat and warm an extremity of a patient wrapped in said sleeve to a temperature sufficient to render veins more prominent and visible to facilitate insertion of an intravenous device.

2. The medical extremity warming system of claim 1, wherein said heating assembly comprises an air channel, a heating unit operatively connected to said air channel to conduct heated air into said air channel, and an exit port disposed on an interior surface of said sleeve and connected to said air channel to permit flow of heated air out of said exit port and about an extremity of a patient wrapped in said sleeve.

3. The medical extremity warming system of claim 2, wherein said heating unit comprises a heating element, a blower unit, and a hose connecting said powered heating unit to said air channel.

4. The medical extremity warming system of claim 3, wherein said air channel comprises a tube being disposed at an outer surface of said sleeve and having a connecting port to detachably connect to said hose.

5. The medical extremity warming system of claim 4, wherein said heating unit comprises at least one of: batteries and a power cord to be plugged into a power outlet.

6. The medical extremity warming system of claim 2, wherein said sleeve comprises a disposable material.

7. The medical extremity warming system of claim 2, wherein said sleeve has a width sufficient to wrap around an arm of a patient and a length sufficient to extend from a shoulder region to a hand region.

8. The medical extremity warming system of claim 2, wherein said first mating member comprises a strip of hook material disposed along an edge of said sleeve and said second mating member comprises a strip of loop material disposed along an opposite edge of said sleeve.

9. The medical extremity warming system of claim 1, wherein said heating assembly comprises a plurality of oxygen-activated heating elements removably or permanently mounted on or in said sleeve.

10. The medical extremity warming system of claim 9, wherein said sleeve comprises at least three pockets configured to each hold one of said heating elements therein.

11. The medical extremity warming system of claim 10, wherein said sleeve comprises at least six of said pockets.

12. The medical extremity warming system of claim 9, wherein said sleeve comprises a disposable material.

13. The medical extremity warming system of claim 9, wherein said sleeve has a width sufficient to wrap around an arm of a patient and a length sufficient to extend from a shoulder region to a hand region.

14. The medical extremity warming system of claim 9, wherein said first mating member comprises a strip of hook material disposed along an edge of said sleeve and said second mating member comprises a strip of loop material disposed along an opposite edge of said sleeve.

15. The medical extremity warming system of claim 1, wherein:
said sleeve comprises a disposable material;
said sleeve has a width sufficient to wrap around an arm of a patient and a length sufficient to extend from a shoulder region to a hand region; and
said first mating member comprises a strip of hook material disposed along an edge of said sleeve and said second mating member comprises a strip of loop material disposed along an opposite edge of said sleeve.

16. The medical extremity warming system of claim 15, wherein:
said heating assembly comprises an air channel, a heating unit operatively connected to said air channel to conduct heated air into said air channel, and an exit port disposed on an interior surface of said sleeve and connected to said air channel to permit flow of heated air out of said exit port and about an extremity of a patient wrapped in said sleeve,
said heating unit comprises a heating element, a blower unit, and a hose connecting said powered heating unit to said air channel;
said air channel comprises a tube being disposed at an outer surface of said sleeve and having a connecting port to detachably connect to said hose; and
said heating unit comprises at least one of: batteries and a power cord to be plugged into a power outlet.

17. The medical extremity warming system of claim 15, wherein:
said heating assembly comprises a plurality of oxygen-activated heating elements removably or permanently mounted on or in said sleeve;
said sleeve comprises a plurality of pockets configured to each hold one of said heating elements therein.

18. A method of using the medical extremity warming system comprising a sleeve being wrappable around an extremity of a patient and configured to retain heat therein; a releasable securing assembly comprising a first mating member at one side of said sleeve and a second mating member at a second side of said sleeve; and a heating assembly being configured to supply heat and warm an extremity of a patient wrapped in said sleeve to a temperature sufficient to render veins more prominent and visible to facilitate insertion of an intravenous device, said method comprising the steps of:
wrapping said sleeve relatively tightly around an extremity of a patient;
releasably connecting said first mating member and said second mating member;
activating said heating assembly and heating the extremity to a temperature sufficient to warm the extremity and thereby render veins more prominent and visible to facilitate insertion of an intravenous device;
waiting a selected amount of time and then releasing and unwrapping said sleeve; and
inserting an intravenous device into a visible vein.

19. A medical extremity warming system comprising:
a sleeve being wrappable around an extremity of a patient and configured to retain heat therein;
a releasable securing assembly comprising a first mating member at one side of said sleeve and a second mating member at a second side of said sleeve; and
a heating assembly comprising an air channel configured to connect to and conduct heated air from a heating unit, and an exit port disposed on an interior surface of said sleeve and connected to said air channel to permit flow of heated air out of said exit port and about an extremity of a patient wrapped in said sleeve to supply heat and warm an extremity of a patient wrapped in said sleeve to a temperature sufficient to render veins more prominent and visible to facilitate insertion of an intravenous device.

20. The medical extremity warming system of claim 19, wherein said air channel comprises a tube being disposed at an outer surface of said sleeve and having a connecting port to detachably connect to said hose.
